(19) United States
(12) Patent Application Publication
(10) Pub. No.: US 2025/0099398 A1
(43) Pub. Date: Mar. 27, 2025

(54) GRAPHENE ACUPOINT HERBAL PATCH (AHP) FOR WARMING UTERUS AND RELIEVING PAIN, METHOD OF PREPARATION AND USE THEREOF

(71) Applicant: Changchun University of Chinese Medicine, Changchun City (CN)

(72) Inventors: Fuchun Wang, Changchun City (CN); Jia Liu, Changchun City (CN); Hailin Jiang, Changchun (CN); Tie Li, Changchun City (CN); Nan Zhang, Changchun City (CN); Jinying Zhao, Changchun City (CN); Wenxuan Cao, Changchun City (CN); Wu Liu, Changchun City (CN); Xuewei Zhao, Changchun City (CN); Lin Wang, Changchun City (CN)

(21) Appl. No.: 18/884,875

(22) Filed: Sep. 13, 2024

(30) Foreign Application Priority Data
Sep. 25, 2023 (CN) 202311235091.2

(51) Int. Cl. A61K 9/70; A61K 31/045; A61K 31/125; A61K 33/44; A61K 36/22; A61K 36/232; A61K 36/235; A61K 36/286; A61K 36/328; A61K 36/533; A61K 36/54; A61K 36/61; A61K 36/65; A61K 36/66; A61K 36/899; A61K 36/9068; A61P 29/00

(52) U.S. Cl. CPC A61K 9/7023; A61K 31/045; A61K 31/125; A61K 33/44; A61K 36/22; A61K 36/232; A61K 36/235; A61K 36/286; A61K 36/328; A61K 36/533; A61K 36/54; A61K 36/61; A61K 36/65; A61K 36/66; A61K 36/899; A61K 36/9068; A61P 29/00

(57) ABSTRACT

A graphene acupoint herbal patch (AHP) for warming uterus and relieving pain, and a method of preparation and use thereof are provided, belonging to the technical field of traditional Chinese medicines (TCMs). The graphene AHP for warming uterus and relieving pain includes a TCM, graphene, a hot melt adhesive, a far-infrared ceramic powder, a heat sensitizer, camphor, and menthol. The graphene AHP for warming uterus and relieving pain produces no acute toxicity, has no skin irritation, and does not induce allergies. In the graphene AHP for warming uterus and relieving pain, the release rate of the small drug molecules tetrahydropalmatine, imperatorin, eugenol, and paeoniflorin is higher than that of an ordinary AHP for warming uterus and relieving pain, thus exhibiting a more obvious analgesic effect.

### CROSS REFERENCE TO RELATED APPLICATION(S)

This application claims priority to Chinese Patent Application No. 202311235091.2, filed with the China National Intellectual Property Administration (CNIPA) on Sep. 25, 2023 and entitled “GRAPHENE ACUPOINT HERBAL PATCH (AHP) FOR WARMING UTERUS AND RELIEVING PAIN, METHOD OF PREPARATION AND USE THEREOF”, which is incorporated herein by reference in its entirety.

### TECHNICAL FIELD

The present disclosure belongs to the technical field of traditional Chinese medicines (TCMs), and particularly relates to a graphene acupoint herbal patch (AHP) for warming uterus and relieving pain, a method of preparation and use thereof.

### BACKGROUND

Since its introduction, graphene materials, with excellent thermal and electrical conductivity properties, have quickly become a hot research topic at home and abroad. Their applications in the medical field mainly focus on drug carriers, photothermal therapy, and targeted therapy. At present, grapheme-based thermal physiotherapy devices have been developed and marketed both domestically and internationally, providing gentle and continuous heat at the active sites to achieve physiotherapy.

In the field of research and application of traditional Chinese medicine (TCM) patches, graphene materials are widely used in the preparation of antipyretic patches, band-aids, and hypotensive patches. In addition, graphene materials also exhibit a strong drug-loading capacity and serve as a carrier material for sustained-release TCM preparations, showing great value and broad prospects. However, there are no relevant records and reports on use of the graphene materials in TCM for the treatment of dysmenorrhea.

### SUMMARY

In view of this, an objective of the present disclosure is to provide a graphene acupoint herbal patch (AHP) for warming uterus and relieving pain, a method of preparation and use thereof. In the present disclosure, the graphene AHP for warming uterus and relieving pain has the effect of analgesic and relieving uterine swelling, and shows a positive effect on treating dysmenorrhea.

To achieve the above objective, the present disclosure provides the following technical solutions.

The present disclosure provides a graphene AHP for warming uterus and relieving pain, including the following raw materials in parts by weight: 25 parts of TCM, 0.1 parts of graphene, 64 parts of hot melt adhesive, 2 parts of far-infrared ceramic powder, 3 parts of heat sensitizer, 3 parts of camphor, 3 parts of menthol; where the TCM includes Rhizoma cyperi, Flos caryophylli, Rhizoma corydalis, Resina olibani, Myrrha, Radix angelicae sinensis, Radix angelicae dahuricae, Herba leonuri, Flos carthami, Fructus foeniculi, Ramulus cinnamomi, Rhizoma zingiberis, and Radix paeoniae alba.

In some embodiments, the Rhizoma cyperi, the Flos caryophylli, the Rhizoma corydalis, the Resina olibani, the Myrrha, the Radix angelicae sinensis, the Radix angelicae dahuricae, the Herba leonuri, the Flos carthami, the Fructus foeniculi, the Ramulus cinnamomi, the Rhizoma zingiberis, and the Radix paeoniae alba are at a mass ratio of 1:1:1:1:1:1:1:1:1:1:1:1:1.

The present disclosure further provides a method for preparing the graphene AHP for warming uterus and relieving pain, including the following steps: crushing the TCM, and mixing the crushed TCM with the graphene to obtain a TCM-graphene mixture; and mixing a melted hot melt adhesive with the far-infrared ceramic powder, the heat sensitizer, the TCM-graphene mixture, the camphor, and the menthol, and applying a resulting mixture on a patch to obtain the graphene AHP for warming uterus and relieving pain.

The present disclosure further provides use of the graphene AHP for warming uterus and relieving pain in the preparation of a drug for treating dysmenorrhea.

Compared with the prior art, the embodiments of present disclosure have the following beneficial effects:

The present disclosure provides a graphene AHP for warming uterus and relieving pain, including a TCM, graphene, a hot melt adhesive, a far-infrared ceramic powder, a heat sensitizer, camphor, and menthol. The graphene AHP for warming uterus and relieving pain produces no acute toxicity, has no skin irritation, and does not induce allergies. Compared with the AHP for warming uterus without adding graphene, the graphene AHP for warming uterus and relieving pain may significantly increase the continuous adhesion time of the AHP to the skin and significantly improve the peeling resistance between the AHP and the skin. The graphene AHP for warming uterus and relieving pain releases the small drug molecules tetrahydropalmatine, imperatorin, eugenol, and paeoniflorin at a higher rate than ordinary AHPs for warming uterus and relieving pain. The graphene AHP for warming uterus and relieving pain exhibits a significant analgesic effect, improves coagulation function, inhibits the secretion of prostaglandins and the expression of COX-2 and IL-1β proteins in vivo, and suppresses the excessive contraction of the uterus and the occurrence of inflammations, thereby treating the dysmenorrhea. After three courses of clinical treatment, the graphene AHP for warming uterus and relieving pain may significantly improve the symptoms of qi stagnation and blood deficiency-type dysmenorrhea, cold coagulation and blood stasis-type dysmenorrhea, and adolescent primary dysmenorrhea, and reduce the patient's visual analogue scale (VAS) and Cox menstrual symptom scale (CMSS).

### BRIEF DESCRIPTION OF THE DRAWINGS

FIG. 1 shows the influence of adding graphene on the permanent adhesion and peeling strength of AHP for warming uterus and relieving pain; note: compared with AHP for warming uterus and relieving pain group, **p<0.01;

FIGS. 2A-2D show Q-t penetration kinetics curves of tetrahydropalmatine, imperatorin, eugenol, and paconiflorin (n=3); FIG. 2A: tetrahydropalmatine; FIG. 2B: imperatorin; FIG. 2C: eugenol; FIG. 2D: paeoniflorin;

FIGS. 3A-3C show the results of an acute toxicity experiment; FIG. 3A: observation results of the behavioral state and facial and oral and nasal secretions of rats; FIG. 3B: body weight changes in rats with damaged skin; C: body weight changes of rats with intact skin;

FIGS. 4A-4D show the results of a skin irritation experiment; FIG. 4A: observation of a single stimulation on intact skin; FIG. 4B: observation of a single stimulation on damaged skin; FIG. 4C: observation of multiple stimulations on intact skin; FIG. 4D: observation of multiple stimulations on damaged skin;

FIGS. 5A-5B shows the results of an allergy experiment; FIG. 5A: sensitization exposure results; FIG. 5B: challenge exposure results;

FIGS. 6A-6C show the inhibitory effect of the AHP on acetic acid-induced writhing in rats; FIG. 6A: statistics of writhing frequencys; FIG. 6B: statistics of writhing latency; FIG. 6C: statistical results of writhing inhibition rate; notes: compared with the blank control group: *p<0.05, ***p<0.001, ns p>0.05; compared with the graphene AHP for warming uterus and relieving pain group: #p<0.05, ##p<0.01, ###p<0.001;

FIGS. 7A-7B show the inhibitory effect of the AHP on hot plate-induced paw withdrawal in rats; FIG. 7A: statistics of the time for rats to withdraw their paws; FIG. 7B: statistics of the percentage increase in pain threshold; notes: compared with the blank control group: **p<0.01, ***p<0.001, ns p>0.05; compared with the graphene AHP for warming uterus and relieving pain group: ##p<0.01, ###p<0.001;

FIGS. 8A-8E show the writhing score, uterine organ coefficients, and prostaglandin secretion in rats with primary dysmenorrhea detected by the graphene AHP for warming uterus and relieving pain; FIG. 8A: statistics of writhing scores; FIG. 8B: statistics of uterine organ coefficients; FIG. 8C: PGE₂ secretion in rat serum; FIG. 8D: PGF_{2α} secretion in rat serum; FIG. 8E: calculation of a ratio of PGE₂ and PGF_{2α}; note: compared with the model group: *p<0.05, **p<0.01, ***p<0.001, ns p>0.05;

FIGS. 9A-9D show the coagulation function in rats with primary dysmenorrhea detected by the graphene AHP for warming uterus and relieving pain; FIG. 9A: detection results of fibrinogen (Fib); FIG. 9B: detection results of prothrombin time (PT); FIG. 9C: detection results of thrombin time (TT); FIG. 9D: detection results of activated partial thromboplastin time (APTT); note: compared with the model group: *p<0.05, **p<0.01, *p<0.001, ns p>0.05;

FIGS. 10A-10C show the blood rheology indicators in rats with primary dysmenorrhea detected by the graphene AHP for warming uterus and relieving pain; FIG. 10A: detection of blood viscosity under high-shear rate conditions; FIG. 10B: detection of blood viscosity under mid-shear rate conditions; FIG. 10C: detection of blood viscosity under low-shear rate conditions; note: compared with the model group: **p<0.01, ***p<0.001, ns p>0.05; and

FIGS. 11A-11C show the influence of the graphene AHP for warming uterus and relieving pain on the expression of inflammation-related proteins in the uterine tissue of rats with primary dysmenorrhea (by Western blot); FIG. 11A: Western blot of COX-2 protein and IL-1β protein; FIG. 11B: statistic of relative expression of COX-2 protein; FIG. 11C: statistic of relative expression of IL-1β protein; note: compared with the model group: **p<0.01, ***p<0.001, ns p>0.05.

### DETAILED DESCRIPTION OF THE EMBODIMENTS

The present disclosure provides a graphene AHP for warming uterus and relieving pain, including the following raw materials in parts by weight: 25 parts of TCM, 0.1 parts of graphene, 64 parts of hot melt adhesive, 2 parts of far-infrared ceramic powder, 3 parts of heat sensitizer, 3 parts of camphor, 3 parts of menthol; where the TCM includes Rhizoma cyperi, Flos caryophylli, Rhizoma corydalis, Resina olibani, Myrrha, Radix angelicae sinensis, Radix angelicae dahuricae, Herba leonuri, Flos carthami, Fructus foeniculi, Ramulus cinnamomi, Rhizoma zingiberis, and Radix paeoniae alba.

In the prescription of the present disclosure, Rhizoma corydalis, Resina olibani, and Myrrha are used together. They are all sovereign medicinal, with bitterness for descending and purging, pungency for dispersing, and warmth for warming and promoting circulation. They work together to invigorate blood circulation and qi flow, reduce swelling, and alleviate pain, addressing the symptoms of pain caused by blockage. For women with symptoms of insufficient qi and blood and pain resulting from inadequate nourishment, the combination of Radix angelicae sinensis, which can nourish blood, activate blood circulation, and relieve pain, and Radix paeoniae alba, which notifies blood, astringes yin, and relieves acute and chronic pain, can replenish blood and vital essence by supplementation and nourishment. When further combined with Flos carthami to activate blood circulation, remove blood stasis, and stimulate menstruation, and Herba leonuri to activate blood circulation, remove blood stasis, and reduce swelling, it not only replenishes blood and qi, but also promotes blood circulation. The above four medicines work together to remove blood stasis and promotes regeneration, serving as minister medicinal for replenishing blood and nourishing vital essence. For the symptoms of cold coagulation and blood stasis that trouble women, Ramulus cinnamomi warms the channels to clear obstruction, dissipates cold to removes impediment; Flos caryophylli warms yang, disperse cold, and relieves pain; Fructus foeniculi disperse cold and relieves pain; Rhizoma zingiberis dispels dampness and cold. When these four medicines are used in combination, they unblock the meridians, disperse adhesions, relax spasms of tendons and muscles, and facilitate joint mobility. They serve as minister medicinal for dispelling cold and relieving pain. Rhizoma cyperi regulates menstruation and relieves pain, while Radix angelicae dahuricae is anti-inflammatory and reduces swelling. They both assist in enhancing the efficacy of the drug, serving as adjuvant medicinal.

The above 13 TCMs are combined with graphene nanoparticles to achieve a triple effects of acupoint stimulation, drug penetration, and targeted release of graphene-loaded drugs. By transdermal administration, the drug can penetrate the skin and directly reach the lesions. With the targeted release effect of graphene, the drug penetration rate and effectiveness are improved, and the multiple effects can be achieved, such as nourishing blood and vital essence, activating blood circulation and removing blood stasis, warming the menstruation and dispersing cold, warming uterus and relieving pain, and anti-inflammatory and reducing swelling.

In the present disclosure, the hot melt adhesive is purchased from Suqian Huateng New Material Technology Co., Ltd., batch number 230811; the far-infrared ceramic powder is purchased from the Fifth Porcelain Factory of Dehua, Fujian Province, batch number 20230501; the heat sensitizer is purchased from Weihai Medicinal Oil Factory in Jishui County, batch number 230726.

In the present disclosure, the Rhizoma cyperi, the Flos caryophylli, the Rhizoma corydalis, the Resina olibani, the Myrrha, the Radix angelicae sinensis, the Radix angelicae dahuricae, the Herba leonuri, the Flos carthami, the Fructus foeniculi, the Ramulus cinnamomi, the Rhizoma zingiberis, and the Radix paeoniae alba are at a mass ratio of preferably 1:1:1:1:1:1:1:1:1:1:1:1:1 in the TCM.

The present disclosure further provides a method for preparing the graphene AHP for warming uterus and relieving pain, including the following steps: crushing the TCM, and mixing the crushed TCM with the graphene to obtain a TCM-graphene mixture; and mixing a melted hot melt adhesive with the far-infrared ceramic powder, the heat sensitizer, the TCM-graphene mixture, the camphor, and the menthol, and applying a resulting mixture on a patch to obtain the graphene AHP for warming uterus and relieving pain.

The present disclosure further provides use of the graphene AHP for warming uterus and relieving pain in the preparation of a drug for treating dysmenorrhea.

In the present disclosure, there are no special restrictions on sources of the raw materials, and conventional commercial products in the art may be used.

The technical solution provided by the present disclosure will be described in detail below with reference to the examples, but they should not be construed as limiting the claimed scope of the present disclosure.

### Example 1

### Preparation of a Graphene AHP for Warming Uterus and Relieving Pain

(1) 1,000 g each of Rhizoma cyperi, Flos caryophylli, Rhizoma corydalis, Resina olibani, Myrrha, Radix angelicae sinensis, Radix angelicae dahuricae, Herba leonuri, Flos carthami, Fructus foeniculi, Ramulus cinnamomi, Rhizoma zingiberis, and Radix paeoniae alba were preciously weighed and crushed into powders, which were mixed well with 100 g of graphene to obtain a TCM-graphene mixture.
(2) 64 g of hot melt adhesive was melted by heating, and 2 g of far-infrared ceramic powder, 3 g of heat sensitizer, 25 g of the TCM-graphene mixture obtained in step (1), 3 g of camphor, and 3 g of menthol were added into a resulting melted pasty hot melt adhesive in sequence to obtain a gel.
(3) The gel was coated on a patch to obtain the graphene AHP for warming uterus and relieving pain containing 10 g of the hot melt adhesive (containing 2.5 g of drug) per patch.

### Example 2

### Drug Loading and Release Efficiency of the Graphene AHP for Warming Uterus and Relieving Pain

### (1) Influence of Graphene on Drug Loading Efficiency of the AHP

The graphene AHP for warming uterus and relieving pain prepared in Example 1 and an AHP for warming uterus and relieving pain (produced by Jilin Guoda Biological Engineering Co., Ltd., supervised by Jilin Ailuokang Pharmaceutical Technology Development Co., Ltd., production batch number: 20220801, and is identical to that of Example 1 except for the absence of graphene) were fixed on a test plate, bonded to a clean polyester film, and allowed to stand for 30 min to allow testing. The free end of the polyester film was opposed (180°), with the free end of the film and the test plate clamped on the upper and lower ends of the testing machine, respectively. The peeling surface should be consistent with the testing machine line. The testing machine continuously peeled at 300 mm/min±10 mm/min, while the automatic recorder recorded a peeling curve, as shown in FIG. 1.

From FIG. 1, it could be seen that the permanent adhesion and peeling strength of the graphene AHP for warming uterus and relieving pain group were significantly higher than those of the AHP for warming uterus and relieving pain group (P<0.01). This indicates that the graphene can significantly increase the continuous adhesion time of the AHP to the skin and significantly improve the peeling resistance between the AHP and the skin.

### (2) Influence of Graphene on the Release Efficiency of the AHP for Warming Uterus and Relieving Pain In Vitro

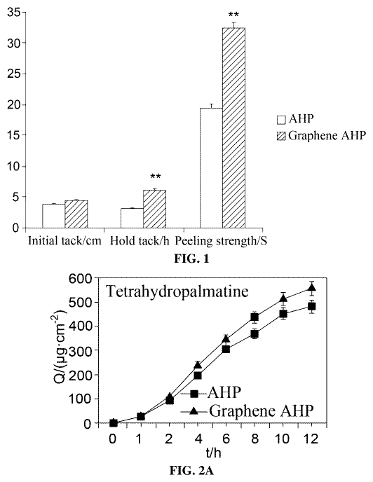

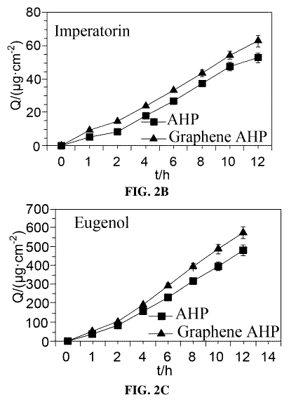

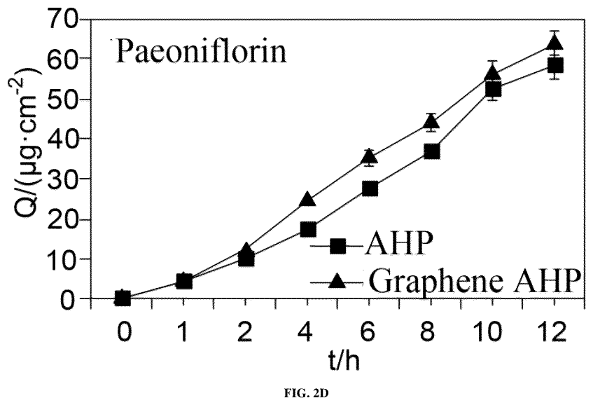

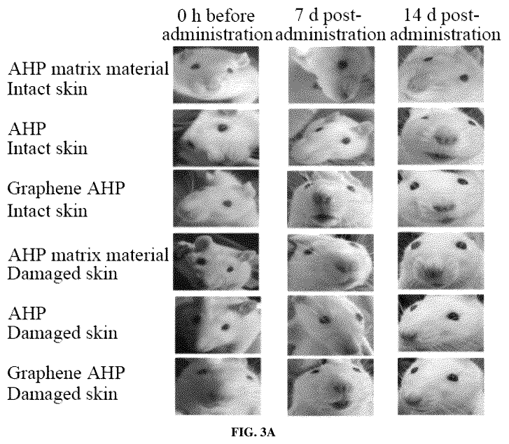

Using Franz diffusion cell method and ultra-high performance liquid chromatography quadrupole time of flight mass spectrometry (UHPLC-Q-TOF-MS) technology, 45 KM mice were randomly divided into 3 groups: an AHP for warming uterus and relieving pain group, a graphene AHP for warming uterus and relieving pain group as per Example 1, and a blank patch group (i.e., patching material group). The mice were sacrificed, and their skin tissues were peeled off and placed at the junction of the supplying pool and the receiving pool of the Franz diffusion cell. An appropriate amount of the graphene AHP for warming uterus and relieving pain and an equal amount of the AHP for warming uterus and relieving pain were separately applied on the excised skin surface of the mouse. The collected receiving solution was qualitatively and quantitatively analyzed using UHPLC-Q-TOF-MS under the intended analysis conditions for tetrahydropalmatine (C₂₁H₂₅NO₄), imperatorin (C₁₆H₁₄O₄), paeoniflorin (C₂₃H₂₈O₁₁), and eugenol (C₁₀H₁₂O₂), and the contents of the above 4 components in the diffusion cell solution and the cumulative permeability Q were calculated. A curve was plotted based on the cumulative permeability Q of the drug versus the sampling time t, and linear regression was conducted on the straight line part to obtain the transdermal flux J. The specific results were shown in FIGS. 2A-2D.

As shown in FIGS. 2A-2D, the addition of graphene could significantly increase the 12-h cumulative release of the 4 components (P<0.01); for the 2 components of tetrahydropalmatine and eugenol after 2 h in the two groups, the release rate of the graphene group was higher than that of the ordinary AHP for warming uterus and relieving pain group. The release rates of 2 components paeoniflorin and imperatorin in the graphene AHP for warming uterus and relieving pain group were higher than those in the ordinary AHP for warming uterus and relieving pain group from 1 h onwards.

The fitting equations obtained based on the data in FIGS. 2A-2D are as follows: for tetrahydropalmatine in the AHP for warming uterus and relieving pain: y=42.299x+15.028, R²=0.9732; for imperatorin: y=4.4972x+0.1371, R²=0.9946; for eugenol: y=8.7648x−13.784, R²=0.9732; for paeoniflorin: y=70.816x−104.48, R²=0.9861. For tetrahydropalmatine in the graphene AHP for warming uterus and relieving pain, y=48.181x+22.818, R²=0.9739; for imperatorin: y=4.8872x+4.2631, R²=0.9993; for eugenol: y=9.6276x−13.432, R²=0.9914; for paeoniflorin: y=85.335x−122.03, R²=0.9886. On the whole, the graphene AHP for warming uterus and relieving pain had a higher release rate than the AHP for warming uterus and relieving pain.

### Example 3

### Research on Safety of the Graphene AHP for Warming Uterus and Relieving Pain

The method for preparing the AHP matrix material in this example was similar to that in Example 1, except that the TCM-graphene mixture was not added.

### (1) Acute Toxicity Test

Thirty Sprague-Dawley (SD) rats were randomly divided into 3 groups, with 10 rats in each group: an AHP matrix material group/an AHP for warming uterus and relieving pain group/a graphene AHP for warming uterus and relieving pain group as per Example 1. Each group was further divided into an intact skin group and a damaged skin group, with 5 rats in each group.

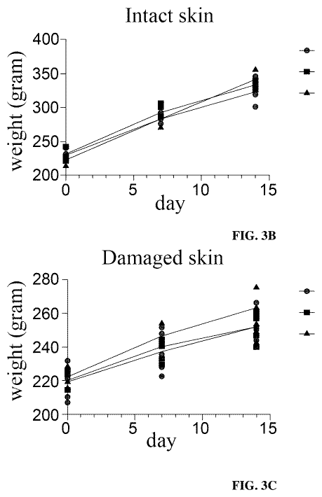

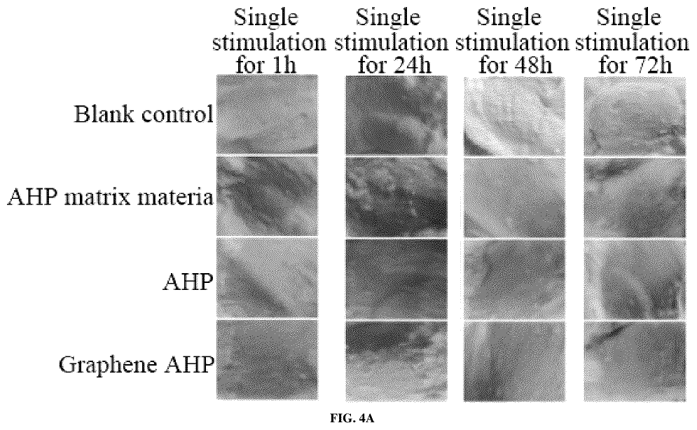

After hair removal on both sides of the back, the rats in the damaged skin group were marked “#” with a syringe needle to induce bleeding on their skin. Each AHP was applied to the left skin of rats in each group, and the right skin was covered with gauze dipped in physiological saline. After 24 h, the test substance remaining on the skin was removed with warm water. The rats were raised normally for 14 d, during which the weight of the rats before administration (day 0) and on days 7 and 14 after administration were recorded separately. The rats' physical signs and other indicators were observed to determine whether there were signs of poisoning, and specific results are shown in FIGS. 3A-3C.

As shown in FIGS. 3A-3C, compared with the blank group, rats still responded to external stimuli after administration, and no death occurred 14 d after administration. No significant changes were found in the fur, eyes, mucous membranes, respiration, and spontaneous activities of the rats in the administration group, and no rats were found to exhibit poisoning symptoms of trembling, tremors, salivation, diarrhea, laziness, lethargy, coma, or even death (FIG. 3A). As shown in FIG. 3B and FIG. 3C, the weight growth rate of the rats in the damaged skin group was slightly lower than that in the intact skin group, the AHP for warming uterus and relieving pain group had no inhibition on the damaged or intact skin group, and the rats given graphene AHP for warming uterus and relieving pain had slower weight gain.

Comprehensive results indicate that the graphene AHP for warming uterus and relieving pain, and materials and updated products thereof do not cause acute toxic reactions in rats, and the overall product safety is guaranteed.

### (2) Skin Irritation Test

After one week of regular feeding, 32 rabbits were randomly divided into 4 groups, 8 rabbits in each group: a blank control group/an AHP matrix material group/an AHP for warming uterus and relieving pain patch group/a graphene AHP for warming uterus and relieving pain group as per Example 1. Each group was further divided into a single stimulation group and a multiple stimulations group, with 4 rabbits in each group.

The rabbit's back was shaved on both sides, with intact skin on one side of the back and damaged skin on the other side. The left and right sides of the same body were compared with each other. For rabbits in the damaged skin group, a syringe needle was used to mark “#” on the skin until blood oozing, and the experiment started after 24 h. Breathable gauze/AHP matrix material/AHP for warming uterus and relieving pain/graphene AHP for warming uterus and relieving pain were separately applied to intact and damaged skin once for 6 h, and the rabbits were visually observed and photographed to record the scores at 1 h, 24 h, 48 h, and 72 h after a single dose. The observation period could be extended upon presence of persistent damages.

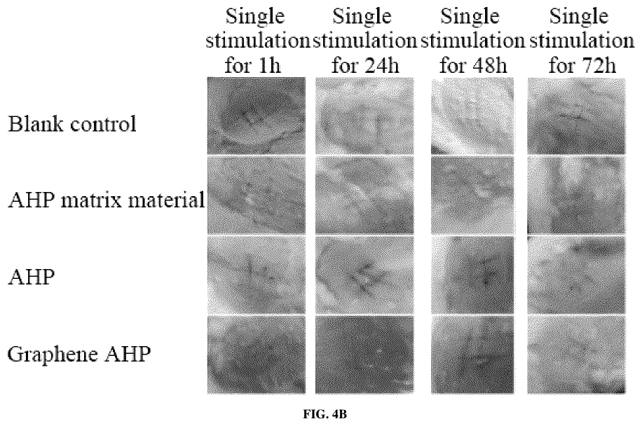

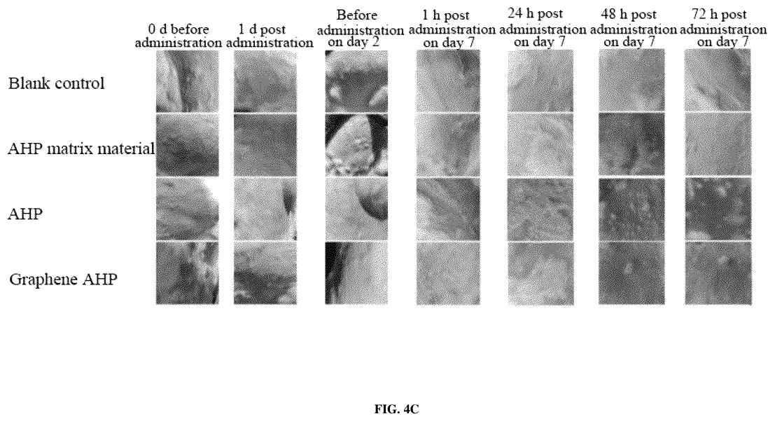

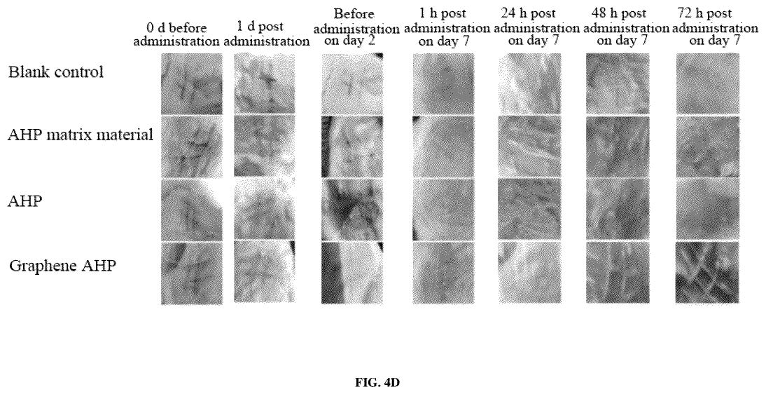

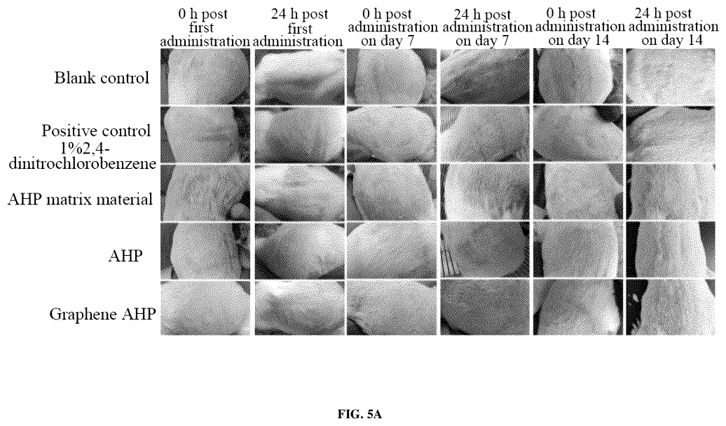

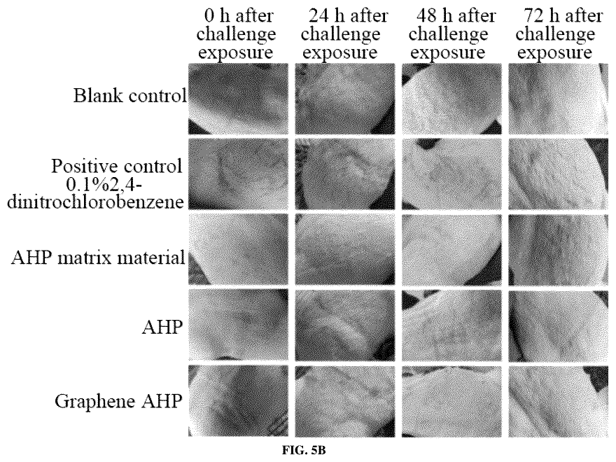

In the repeated irritation experiment, breathable gauze/AHP matrix material/AHP for warming uterus and relieving pain/graphene AHP for warming uterus and relieving pain were separately applied to intact and damaged skin for 6 h each time, once a day for 7 d. The rabbits were visually observed and photographed to record the scores at 1 h, 24 h, 48 h, and 72 h before administration (0 d) and after administration on day 1, before administration on day 2, and after the last administration on day 7. The specific results are shown in FIGS. 4A-4D.

As shown in FIGS. 4A-4D, after 1 week of drug administration on intact and damaged rabbit skin, there was no edema or erythema in the blank control group within 1 h of removing the drug, only mild erythema occurred in the AHP for warming uterus and relieving pain group without edema, and there was no irritation observed in the graphene AHP for warming uterus and relieving pain group. In the repeated skin irritation experiment and the skin single irritation experiment on rabbits, no erythema or edema occurred at all application sites in 16 rabbits. The observed results were consistent within the next 24 h, 48 h, and 72 h, with no significant changes, indicating that the graphene AHP for warming uterus and relieving pain in Example 1 is non-irritating.

### (3) Skin Allergy Test

25 guinea pigs were randomly divided into 5 groups, 5 in each group: a blank control group/a positive drug group/an AHP matrix material group/an AHP for warming uterus and relieving pain group/a graphene AHP for warming uterus and relieving pain group as per Example 1.

The guinea pigs were shaved on both sides of their backs, and gauze or various patches were applied to the left skin of the guinea pigs in each group for 6 h. The positive drug group was applied with 1% 2,4-dinitrochlorobenzene solution with cotton swabs, and administration was repeated on the 7th and 14th day. Skin allergies were observed and scored at 0 h and 24 h after each dose. On the 28th day, the guinea pigs received challenge administration. The right skin of the guinea pigs in each group was covered with gauze or various patches for 6 h, and the positive drug group was applied with 0.1% 2,4-dinitrochlorobenzene solution with cotton swabs. Skin allergies were observed and scored again at 0 h, 24 h, 48 h, and 72 h after administration, and the guinea pigs were closely observed for severe systemic allergic reactions such as asthma, unsteady standing, or shock. The specific results are shown in FIGS. 5A-5B.

As shown in FIGS. 5A-5B, FIG. 5A shows the results of sensitization exposure. No skin allergies such as erythema and edema were found in the blank control group. The observations of the AHP for warming uterus and relieving pain group, the graphene AHP for warming uterus and relieving pain group, and the AHP matrix material group were similar to those of the blank group, indicating that the medicinal ingredients or patch materials did not cause allergic reactions. The positive control group produced mild erythema but no edema or severe allergic symptoms compared with the administration group. The guinea pigs in each group also did not experience systemic allergic reactions, such as asthma or unsteadiness. These results prove that the AHP for warming uterus and relieving pain does not cause allergic reactions in animals and is a relatively safe therapy.

The challenge exposure results are shown in FIG. 5B. Similar to the sensitization exposure results, during the 72-h observation period after stimulation, there were no abnormalities in the general conditions of the AHP matrix material group, AHP for warming uterus and relieving pain group, and graphene AHP for warming uterus and relieving pain group, as compared with the blank control group. That is, there were no symptoms such as urination, coughing, sneezing, or shortness of breath, and no severe systemic allergic reactions such as unsteady standing, asthma, movement disorders, shock, and spasms. The above results show that the graphene AHP for warming uterus and relieving pain in Example 1 has basically no sensitization reactions on guinea pig skin.

### Example 4

### Effect of Graphene AHP for Warming Uterus and Relieving Pain on Primary Dysmenorrhea

### (1) Influence of the Graphene AHP for Warming Uterus and Relieving Pain on Hot Plate-Induced Pain in Rats

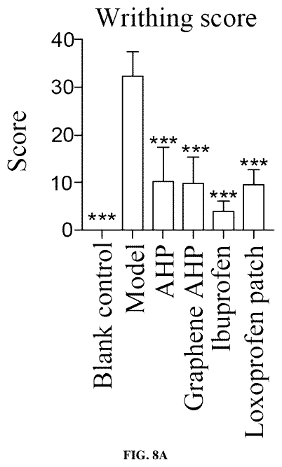

Forty SD rats were randomly divided into 4 groups, with 5 rats in each group, including a blank group, an AHP for warming uterus and relieving pain group, a graphene AHP for warming uterus and relieving pain group, and a positive control group (loxoprofen patch group). After abdominal hair removal of rats, except for the blank group, each group of patches was applied to the abdomen of SD rats for 4-6 h, wrapped and fixed with medical tape, and administered continuously for 7 d. Rats in the blank group were wrapped around the abdomen with tape. After the last administration, the rats were placed into the hot plate at 52° C. twice, 30 min and 60 min, respectively. The time taken for the rats to withdraw their hind paws was recorded. The average time was used as the pain threshold. The pain threshold of the rats in the blank group was used as a baseline pain threshold. The percentage increase in pain threshold of rats in each group was calculated at different time points after administration according to the formula: Percentage increase in pain threshold (%)=[(pain threshold after administration−baseline pain threshold)/baseline pain threshold]×100% (If the rats showed no pain response within 60 s on the hot plate instrument, the rat was taken out immediately and the time was recorded as 60 s. The pain threshold was calculated separately before and after administration). The specific results are shown in FIGS. 7A-7B.

FIG. 7A, which was plotted according to the sorted data collected at the 30-min detection point, shows that compared with the blank control group, the rats in the other three groups after administration had a prolongation in the time taken for paw withdrawal after heat stimulus, where the rats in the graphene AHP for warming uterus and relieving pain group had the longest time among the four groups. When tested at the 60-min test point, the pain threshold of the rats in the drug administration group increased in different ways. This indicates that graphene AHP for warming uterus and relieving pain had a better effect than AHP for warming uterus and relieving pain and loxoprofen patch, leading to a more significant improvement in the pain threshold of SD rats. FIG. 7B shows that graphene AHP for warming uterus and relieving pain had the highest pain threshold improvement percentage in rats. This further confirms that the analgesic effect of graphene AHP for warming uterus and relieving pain in Example 1 is superior to that of AHP for warming uterus and relieving pain.

### (2) Influence of the Graphene AHP for Warming Uterus and Relieving Pain on Acetic Acid-Induced Pain in Rats

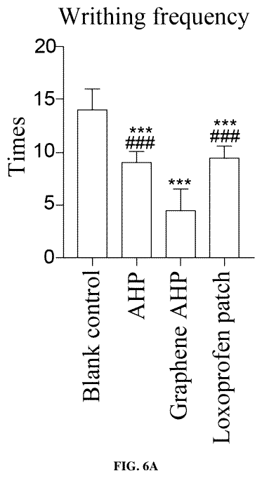

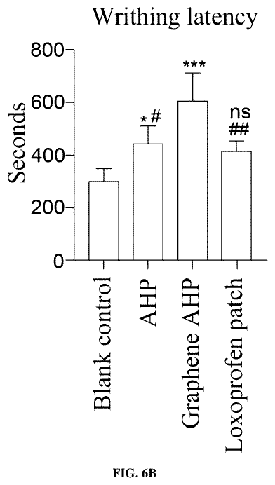

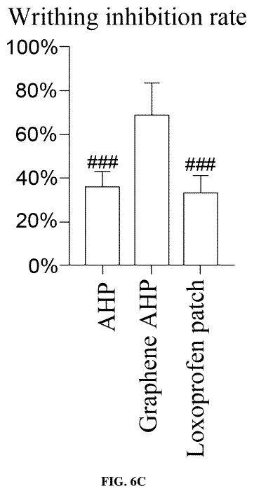

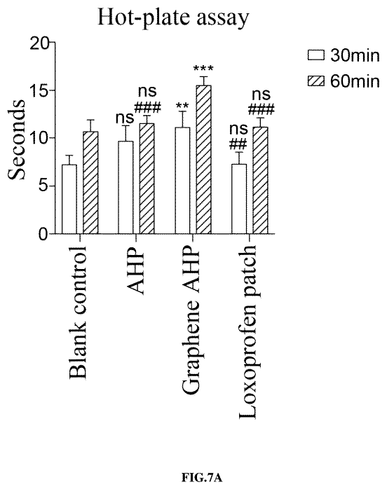

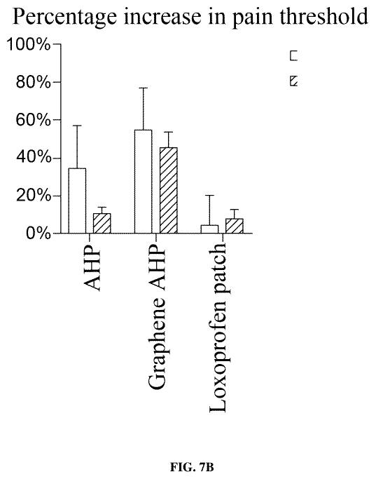

Forty SD rats were randomly divided into 4 groups, with 5 rats in each group, including a blank group, an AHP for warming uterus and relieving pain group, a graphene AHP for warming uterus and relieving pain group, and a positive control group (loxoprofen patch group). After abdominal hair removal of rats, except for the blank group, each group of patches was applied to the abdomen of SD rats for 4-6 h, wrapped and fixed with medical tape, and administered continuously for 7 d. Rats in the blank group were wrapped around the abdomen with tape. Rats were intraperitoneally injected with 2% glacial acetic acid solution (3 mL/kg) 1 h after the last dose. The timing was started after the injection while the latency(s) for the first writhing response and the writhing frequencys within 15 min were observed in the rat after injection of glacial acetic acid. The writhing inhibition rate was calculated according to the formula: writhing inhibition rate (%)=(mean writhing frequencys in model group−mean writhing frequencys in administration group)/mean writhing frequencys in model group×100%. The specific results are shown in FIGS. 6A-6C.

In FIGS. 6A-6C, FIG. 6A shows the statistics of writhing frequencys in rats. Compared with the blank control group, the number of writhes in the three groups of SD rats applied with AHP for warming uterus and relieving pain, graphene AHP for warming uterus and relieving pain, and loxoprofen patch was significantly lower than that of the blank group (p<0.001), indicating that the three groups of patches could significantly inhibit the writhing frequencys in rats after administration. The graphene AHP for warming uterus and relieving pain group induced the lowest writhing frequencys in rats, showing the strongest inhibitory effect. FIG. 6B shows the writhing latency of SD rats. The writhing latency of both AHP for warming uterus and relieving pain and loxoprofen patch was longer than that of the blank group. The writhing latency of rats in the graphene AHP for warming uterus and relieving pain group was significantly longer than that of the other 2 groups, confirming the improved efficacy of the modified AHP. As shown in FIG. 6C, the writhing inhibition rate of AHP for warming uterus and relieving pain was about double that of the other two groups of patches. It is sufficient to conclude that the analgesic effect of graphene-added AHP for warming uterus and relieving pain has been further enhanced.

(3) Therapeutic Effect of the Graphene AHP for Warming Uterus and Relieving Pain on Rats with Primary Dysmenorrhea

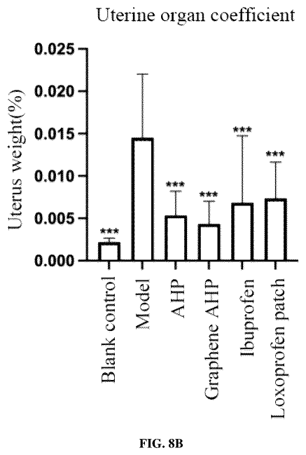

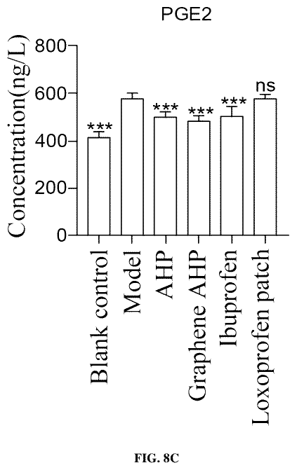

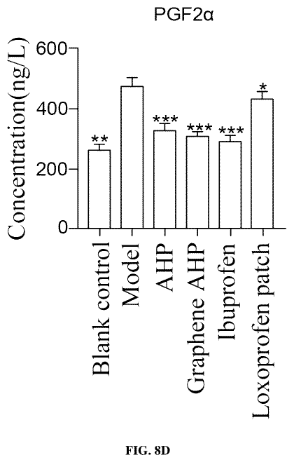

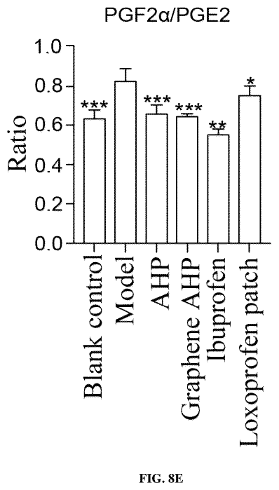

Thirty SD rats were randomly divided into 6 groups, including a blank group, a model group, an AHP for warming uterus and relieving pain group, a graphene AHP for warming uterus and relieving pain group, and a positive control group (ibuprofen granule group and loxoprofen patch group), 5 rats in each group. After adaption, the animal model of primary dysmenorrhea was prepared by subcutaneous injection of estradiol benzoate in the thigh combined with intraperitoneal injection of oxytocin, The modeling dose of estradiol benzoate was 0.5 mg/animal on the 1st and 10th days separately, and 0.2 mg/animal from the 2nd to 9th days. The AHP for warming uterus and relieving pain group and the graphene AHP for warming uterus and relieving pain group were given AHP stimulation at Shenque point and Sanyinjiao point for 4 h to 6 h every day. The loxoprofen patch group was given AHP stimulation at Shenque point for 4 h to 6 h. The patches were wrapped and fixed with medical tape, and the remaining three groups were also wrapped with tape. The ibuprofen granule group was administered by gavage. On the 10th day, 1 h after the last subcutaneous injection of estradiol benzoate into the thighs of SD rats, 2 U of oxytocin per rat was injected intraperitoneally, while the blank group was injected with an equal amount of normal saline. The rat's writhing and arching back were observed and recorded to assign scores. After the observation, the rats were anesthetized and their blood and uterine tissue were collected. Part of the blood was centrifuged to obtain serum for enzyme-linked immunosorbent assay (ELISA) analysis of contents of prostaglandin F_{2α} and prostaglandin E₂, while the other part of the blood was used for hemorrheology testing and coagulation function testing. The uterine tissue was weighed to calculate the organ coefficient, and the total protein extracted from the uterine tissue was subjected to for Western blot analysis to detect the expression of inflammation-related proteins. The specific results are shown in FIG. 8A to FIG. 11C.

As shown in FIG. 8A, the writhing and arching of the back in the model group were very severe. Both AHP for warming uterus and relieving pain and graphene AHP for warming uterus and relieving pain had desirable treatment effects, but there was no significant difference between the two groups. FIG. 8B demonstrates that the uterine coefficient of the model group increased significantly compared with the blank group (due to swelling), while the weight of the rat uterus in the AHP for warming uterus and relieving pain group and the graphene AHP for warming uterus and relieving pain group each decreased significantly compared with the model group. FIG. 8C to FIG. 8E show the secretion of prostaglandins in serum: the secretion levels of PGE₂ and PGF_{2α} in the model group were significantly increased compared with those in the blank group. The two patches showed significant inhibitory effects, where the graphene AHP for warming uterus and relieving pain had a regulatory effect on PGF_{2α} better than that of the oral positive drug ibuprofen.

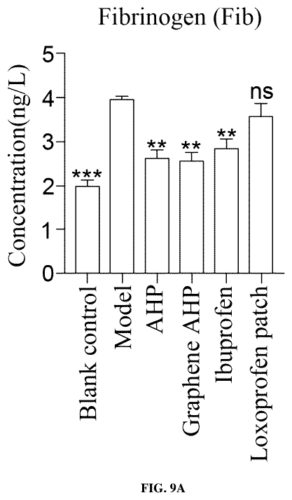

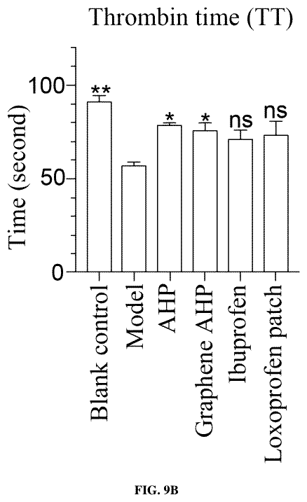

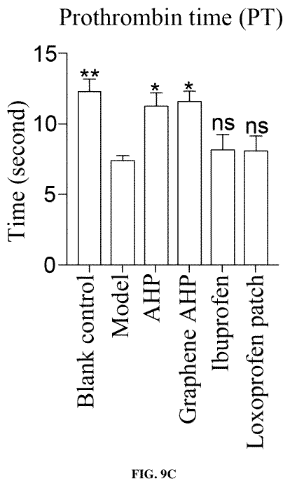

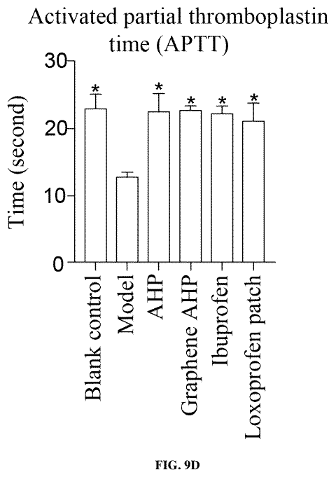

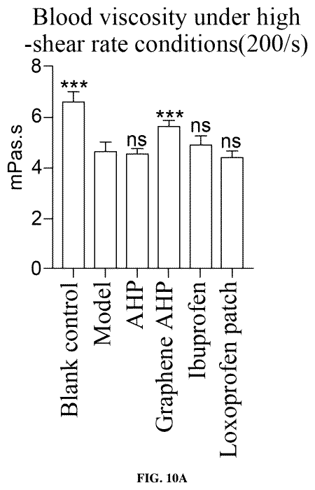

FIGS. 9A-9D show that the coagulation function of the rats in the model group was significantly increased compared with the rats in the blank group (pathological level). The coagulation function of the rats after AHP for warming uterus and relieving pain and graphene AHP for warming uterus and relieving pain treatments returned to normal levels, showing comparable therapeutic effect between the two groups.

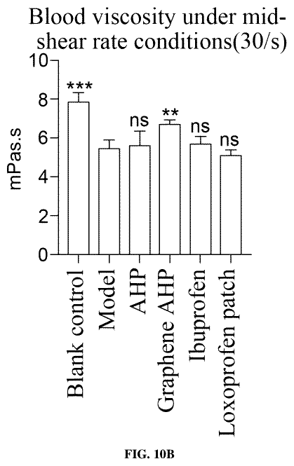

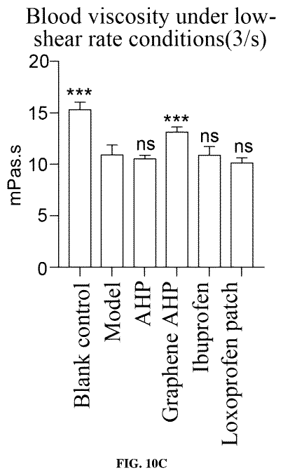

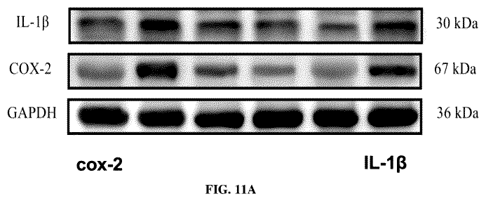

FIGS. 10A-10C show that the blood viscosity of the model group was lower than that of the blank group, with only the therapeutic effect of graphene AHP for warming uterus and relieving pain showing a significant treatment effect.

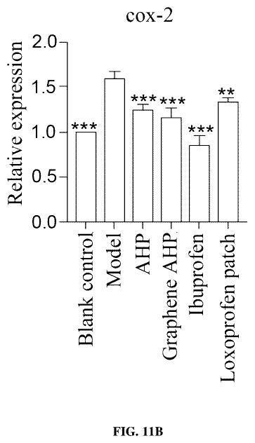

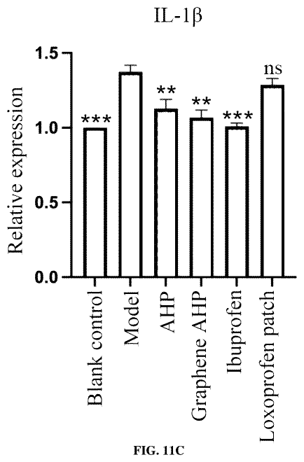

FIGS. 11A-11B show that the expression of COX-2 and IL-1β proteins in the model group was significantly up-regulated. The AHP for warming uterus and relieving pain and graphene AHP for warming uterus and relieving pain could down-regulate the expression of these two proteins, indicating that both patches can inhibit the occurrence of primary dysmenorrhea by inhibiting inflammatory reactions.

### Example 5

### Clinical Study of the Graphene AHP for Warming Uterus and Relieving Pain on Primary Dysmenorrhea

### 1. Clinical Study of the Graphene AHP for Warming Uterus and Relieving Pain in Treating Qi Stagnation and Blood Stasis-Type Primary Dysmenorrhea

### (1) Diagnostic Criteria

### 1) Diagnostic Criteria in Western Medicine

The diagnostic criteria for primary dysmenorrhea were based on the “Clinical Guidelines for Primary Dysmenorrhea” formulated by the Canadian Association of Obstetricians and Gynecologists in 2017: (i) menstrual pain occurs a few hours before or after menstruation in women of childbearing potential and can last for 2-3 d; (ii) the pain is generally in the middle of the lower abdomen, can also radiate to both sides of the lower abdomen, waist and thighs, presenting as cramping or dull pain; (iii) the patient may be accompanied by symptoms such as diarrhea, nausea, vomiting, fatigue, dizziness, headache, which may even affect normal work and life; (iv) B-ultrasound and gynecological examination: organic pelvic diseases are excluded.

### 2) Diagnostic Criteria in TCM

The diagnostic criteria were formulated based on the “Diagnostic and Efficacy Standards for Gynecological Diseases and Syndrome of TCM”:

Diagnostic criteria for dysmenorrhea in TCM: (i) periodic lower abdominal pain during or before and after menstruation, pain reaching the lumbosacral region, and even fainting; (i) symptoms are more common in young unmarried women; (i) organic pelvic diseases are excluded.

Syndrome differentiation criteria for primary dysmenorrhea (qi stagnation and blood stasis type): (i) primary symptoms: distension and pain in the lower abdomen before or during menstruation that refuses to be pressed; (ii) secondary symptoms: possibly accompanied by low menstrual blood volume; poor blood circulation; dark purple blood with clots; premenstrual breast swelling and pain; chest tightness and discomfort; (iii) tongue and pulse: the tongue shows dark purple or has ecchymosis, and the pulse is taut or rough; basis for syndrome differentiation: there are primary symptoms and any 2 or more secondary symptoms at the same time, combined with the tongue and pulse examination.

### (2) Observation Indicators of Therapeutic Effect

### 1) VAS

The VAS adopts a 10-point line scale, where 0 represents no pain and 10 represents extreme pain.

### 2) CMSS

The CMSS consists of 18 items during menstruation, including: lower abdominal pain, nausea, vomiting, loss of appetite, headache, waist and lower back pain, fatigue, dizziness, diarrhea, facial complexion changes, stomach pain, leg pain, facial flushing, body pain, depression (low mood, depression, preference for quiet), irritability (diarrhea caused by emotional changes, such as tension, anger), neuroticism (more sensitive to people and things than usual), and insomnia. Each item is scored on a 5-point scale based on severity and duration.

Scoring for each item includes severity and duration: (i) severity: 0—no discomfort, 1—mild discomfort, 2—moderate discomfort, 3—severe discomfort, 4—extremely severe; (ii) duration: 0—none, 1—lasting for <3 h, 2—lasting for 3-7 h, 3—lasting for 7-24 h, 4—lasting for >24 h.

### 3) TCM Symptom Score of Dysmenorrhea

According to the “Guiding Principles for Clinical Research of New TCMs”, the TCM syndrome (qi stagnation and blood stasis type) scoring standards were formulated, including primary symptoms, secondary symptoms, and tongue and pulse:
(i) Primary symptoms (distension and pain in the lower abdomen before or during menstruation with refusal to press) 0 points: no lower abdominal distension and pain; 2 points: noticable lower abdominal pain before or during menstruation, but able to continue working, no systemic symptoms; 4 points: unbearable lower abdominal pain before or during menstruation, relieved after taking analgesic measures; 6 points: unbearable lower abdominal pain before or during menstruation, restless, seriously affecting work and daily life; must rest in bed, no obvious relief after taking analgesic measures.
(ii) Secondary symptoms: a. small menstrual blood volume 0 points: none, 1 point: yes; b. Poor blood circulation 0 points: no, 1 point: yes; c. Dark purple blood with clots: 0 points: no, 1 point: yes; d. Premenstrual breast swelling and pain: 0 points: no, 1 point: yes; e. Chest tightness and discomfort: 0 points: no, 1 point: yes.
(iii) Tongue and pulse: a. Purple tongue or ecchymosis 0 points: no, 1 point: yes; b. Taut pulse 0 points: no, 1 point: yes.

The TCM symptom scores of dysmenorrhea were evaluated in the two groups of patients after treatment.

### 4) Criteria for Judging Overall Efficacy

Efficacy determination criteria: formulated with reference to the “Guiding Principles for Clinical Research of New TCMs”.

Efficacy index(n)=[(points before treatment−points after treatment)÷points before treatment]×100%; points=VAS score+CMSS score+TCM symptom score.
(i) cured: n≥95%, abdominal pain and other symptoms disappear; (ii) markedly effective: 95%>n≥70%, significant reduction in abdominal pain, improvement in other symptoms; (iii) effective: 70%>n≥30%, slight reduction in abdominal pain, improvement in other symptoms; (iv) ineffective: n<30%, no significant change in abdominal pain and other symptoms; overall effective rate=cure rate+significant effective rate+effective rate.

### (3) Case Criteria

Inclusion criteria: (i) meeting the diagnostic criteria of Western medicine and TCM for primary dysmenorrhea (PD); (ii) aged 16-35 years old; (iii) menstrual cycle regularity: 21-35 d/3-7 d; (iv) VAS≥4 points; (v) CMSS>10 points; (vi) no history of severe drug allergy; (vii) voluntarily signing the informed consent form; note: those who meet all the above creteria at the same time can be included.

Exclusion criteria: (i) presence of pelvic organic lesions, such as adenomyosis; (ii) presence of serious primary diseases such as liver and kidney and malignant tumors; (iii) taking birth control pills or using intrauterine devices (IUDs); (iv) occurrence of severe mental illness, such as anxiety; (v) history of severe drug allergy; (vi) those whose VAS and CMSS do not meet the above requirements and cannot cooperate with treatment; note: those who meet any of the above criteria can be excluded.

Dropout and elimination criteria: (i) serious adverse reactions or worsening of the condition during the trial; (i) development of other diseases or use of other drugs that affect the judgment of efficacy and safety during the trial; (iii) occurrence of special physiological changes, such as pregnancy during the trial; (iv) poor compliance, non-compliance, non-cooperation with the doctor, or withdrawal due to various reasons; (v) participation in other clinical trials during the trial; (vi) those who are un-blinding midway due to various reasons; note: those who meet any of the above are dropped out.

### (4) Case Grouping

According to the diagnostic criteria of dysmenorrhea of TCM and Western medicine and the dialectical criteria of qi stagnation and blood stasis-type primary dysmenorrhea, 60 patients with primary dysmenorrhea were selected. According to the simple random method (lottery method), the 60 patients with qi stagnation and blood stasis-type primary dysmenorrhea were randomly assigned to the graphene AHP for warming uterus and relieving pain group (experimental group) and the placebo AHP group (control group, ingredients included buckwheat flour, honey, and excipient). One patient dropped out of each group during the implementation, that is, 29 patients were actually included in each of the experimental group and the control group. The age and disease course of the patients in the experimental group and the control group were comparable, with no statistically significant differences, allowing for further research.

The operations of the experimental group and the control group were the same: patients were placed in a supine position, with their lower abdomen and lower limbs exposed. After routine disinfection of the local skin, the graphene AHP for warming uterus and relieving pain or placebo AHP was applied at the Guanyuan acupoint, uterus (bilaterally), Sanyinjiao acupoint (bilaterally), and Xuehai acupoint (bilaterally) for 5 h each time.

Both groups started treatment 7 d before menstruation, 1 time a day, for 10 consecutive days, with 1 menstrual cycle as one course of treatment, and continued treatment for 3 courses. After the 3 courses of treatment, the effects were evaluated (including VAS, CMSS, and dysmenorrhea TCM symptom score), and the results were statistically analyzed using SPSS 26.0 software.

### (5) Comparison of VAS Between Groups

The comparison results of the VAS between groups are shown in Table 1.

TABLE 1
Comparison of VAS between groups

| | Experimental | | | |
|---|---|---|---|---|
| | group | Control | Z(t) | P |
| First course | 3.97 ± 1.86 | 6.07 ± 1.39 | −4.881^{#} | 0.000^{♦} |
| of treatment | | | | |
| Second course | 2 (2.00) | 6 (3.00) | −5.618* | 0.000^{•} |
| of treatment | | | | |
| Third course | 2 (2.00) | 7 (3.50) | −5.967* | 0.000^{•} |
| of treatment | | | | |

Notes:
^{#}represents the t value obtained by two independent samples in t test,
*represents the Z value obtained by two independent samples in rank sum test,
^{••} represents the P value obtained by two independent samples in t test, and
^{•}represents the P value obtained by two independent samples in rank sum test.

As shown in Table 1, after the second and third courses of treatment, the differences between the two groups were statistically significant. This indicates that treatment in the experimental group can significantly reduce the VAS scores of patients with qi stagnation and blood stasis-type primary dysmenorrhea.

### (6) Comparison of CMSS Between Groups

The comparison results of the CMSS between groups are shown in Table 2.

TABLE 2
Comparison of CMSS between groups

| | Experimental | | | |
|---|---|---|---|---|
| | group | Control | Z | P |
| First course of treatment | 24 (21.00) | 46 (43.00) | −3.036 | 0.002 |
| Second course of treatment | 17 (17.50) | 45 (46.50) | −4.084 | 0.000 |
| Third course of treatment | 10 (15.50) | 45 (46.50) | −4.739 | 0.000 |

As shown in Table 2, the differences between the experimental group and the control group were statistically significant in the first, second and third courses of treatment. This indicates that treatment of the experimental group could significantly reduce the CMSS score of qi stagnation and blood stasis-type primary dysmenorrhea, which was significantly higher than that of the control patients.

### (7) Comparison of Dysmenorrhea TCM Symptom Scores Between Groups

The comparison results of dysmenorrhea TCM symptom scores between groups are shown in Table 3.

TABLE 3
Comparison of dysmenorrhea TCM
symptom scores between groups

| | Experimental | | | |
|---|---|---|---|---|
| | group | Control | Z | P |
| First course of treatment | 4 (2.00) | 8 (2.50) | −5.509 | 0.000 |
| Second course of treatment | 3 (2.00) | 7 (1.50) | −5.887 | 0.000 |
| Third course of treatment | 2 (1.00) | 7 (2.00) | −5.914 | 0.000 |

As shown in Table 3, the differences between the experimental group and the control group were statistically significant in the first, second and third courses of treatment. This indicates that treatment in the experimental group could significantly reduce the TCM symptom scores of patients with qi stagnation and blood stasis-type primary dysmenorrhea.

### (8) Overall Clinical Effective Rate

The comparison results of the overall clinical effective rate between the experimental group and the control group are shown in Table 4.

TABLE 4
Comparison of overall clinical effective rates of two groups (cases)

| | | | | | Overall | | |
|---|---|---|---|---|---|---|---|
| | | Markedly | | | effective | | |
| Group | Cure | effective | Effective | Ineffective | rate | Z | P |
| Experimental group | 3 | 14 | 7 | 5 | 82.76% | −3.729 | 0.000 |
| Control | 0 | 0 | 7 | 22 | 24.14% | | |

As shown in Table 4, the difference in the overall clinical effective rate between the two groups was statistically significant. This indicates that the graphene AHP for warming uterus and relieving pain had a high overall effective rate in treating the qi stagnation and blood stasis-type primary dysmenorrhea.

### 2. Clinical Study of the Graphene AHP for Warming Uterus and Relieving Pain in Treating Cold Coagulation and Blood Stasis-Type Primary Dysmenorrhea

### (1) Diagnostic Criteria

### 1) Diagnostic Criteria in Western Medicine

The Western medicine diagnostic criteria for cold coagulation and blood stasis-type primary dysmenorrhea were the same as the diagnostic criteria for qi stagnation and blood stasis-type primary dysmenorrhea.

### 2) Diagnostic Criteria in TCM

The diagnostic criteria referred to the relevant content in the textbook “TCM Gynecology” and “TCM Gynecological Diseases and Syndrome Diagnosis and Efficacy Standards” [66].

Primary symptoms: cold pain in the lower abdomen during menstruation or before menstruation, pain with refusal to press, and pain that is relieved by heat. Secondary symptoms: (i) scanty menstrual blood; (ii) dark menstrual blood; (iii) blood clots in menstrual blood; (iv) chills and cold limbs; (v) pale complexion; (vi) profuse cold sweat; (vii) nausea and vomiting; (viii) diarrhea. Tongue and pulse: dark tongue, or petechiae on the edges, white coating, deep and tight pulse. Notes: the primary symptoms are essential, and the secondary symptoms must include two or more, combined with the tongue and pulse examination for diagnosis.

### (2) Efficacy Observation Indicators

### 1) VAS

The VAS adopted a 10-point scale line, where 0 represents no pain and 10 represents extreme pain.

### 2) CMSS

The CMSS was the same as that of qi stagnation and blood stasis-type primary dysmenorrhea.

### 3) TCM Symptom Score of Dysmenorrhea

The scores were summed for primary symptoms, secondary symptoms, tongue symptoms, and pulse symptoms, respectively:
(i) Primary symptoms: 0 points: no cold pain or tingling pain in the lower abdomen; 2 points: slight cold pain or tingling pain in the lower abdomen, without affecting work or life; 4 points: obvious cold pain or tingling pain in the lower abdomen that affects work or life; 6 points: unbearable cold pain or tingling pain in the lower abdomen, requiring bed rest.
(ii) Secondary symptoms: 2 points: dark menstrual blood; 2 points: small menstrual blood volume; 2 points: chills and cold limbs; 2 points: pale complexion; 2 points: dripping cold sweat; 2 points: nausea and vomiting; 2 points: diarrhea.
(iii) Tongue symptoms: 2 points: dark tongue with white coating; 0 points: normal tongue quality.
(iv) Pulse symptoms: 2 points: deep and taut pulse; 0 points: flat pulse.

The TCM symptom scores of dysmenorrhea were evaluated in the two groups of patients after treatment.

### 4) Criteria for Judging Overall Efficacy

The criteria referred to the “Guiding Principles for Clinical Research of New TCMs” specified in 2002. The score reduction rate was calculated and evaluated using the following method, and then the clinical efficacy of each patient was evaluated based on the score reduction rate.

Dysmenorrhea symptom score reduction rate=(pre-treatment composite score−post-treatment composite score)/pre-treatment composite score×100%;

Composite score=VAS score+CMSS score+TCM symptom score.

Clinical cured: dysmenorrhea symptom score reduction rate ≥95%; markedly effective: 70%≤dysmenorrhea symptom score reduction rate <95%; effective: 30%≤dysmenorrhea symptom score reduction rate <70%; ineffective: the dysmenorrhea symptom score reduction rate <30%; overall effective rate=[(cured cases+markedly effective cases+effective cases)/total cases]×100%.

### (3) Case Criteria

Inclusion criteria: (i) meeting the above diagnostic criteria of TCM and Western medicine and belonging to primary dysmenorrhea (cold coagulation and blood stasis type); (ii) aged 16-35 years old; (iii) normal menstrual cycle (28±7 d); (iv) dysmenorrhea VAS score ≥4 points; (v) CMSS score >10 points; (vi) no other treatments received for at least one menstrual cycle before entering this study, and who no use of sedatives, analgesics, or hormonal drugs within 2 weeks before treatment; (vii) patient agrees to participate in this study and voluntarily obeys the randomization; (viii) no history of drug allergy; notes: the above 8 inclusion criteria must be met at the same time to be included in the observation.

Exclusion criteria: (i) patients who do not meet the inclusion criteria; (ii) age <16 years or >35 years; (iii) patients with secondary dysmenorrhea such as endometriosis, adenomyosis, uterine fibroids, and pelvic inflammatory disease; (iv) women who are pregnant, planning pregnancy or breastfeeding; (v) patients with serious primary diseases such as cardiovascular and cerebrovascular, liver, kidney and hematopoietic systems and mental disorders; (vi) patients with ulceration or infection at or near the acupoint where the application is applied; (vii) those who have a history of severe allergies and are allergic to more than 2 types of food or drugs. Note: Anyone who meets any of the above conditions was excluded.

### Dropout Criteria:

(i) during the trial, if the subject has an allergic reaction or serious adverse event deemed necessary to stop the trial by the doctor; (ii) during the trial, if the subject develops other diseases that affect the judgment of efficacy and safety; (iii) during the trial, if the subject has special physiological changes such as pregnancy, that make it inappropriate to continue the study; (iv) during the trial, if the subject develops other diseases that affect the determination of efficacy; (v) the subject proposes to withdraw from the clinical study midway; (vi) the subject has poor compliance, is unwilling or disobeys the treatment, and requests to withdraw from the trial and is stopped by the physician; (vii) although the subject has not explicitly request to withdraw from the trial, he/she no longer receive medication and monitoring and is lost to follow-up; (viii) those who use other treatments or drugs that are prohibited from being used together will affect the determination of effectiveness.

Exclusion criteria: (i) those are included due to misdiagnosis; (ii) those who meet the inclusion criteria but have not taken any medication since inclusion; (iii) combined use of drugs outside the prescribed scope, especially drugs that have a greater impact on test results and affect the effectiveness determination; (iv) participants in other clinical trials during the trial; (v) those who have no evaluation records after inclusion in the trial.

### (4) Case Grouping

According to the diagnostic criteria of dysmenorrhea of TCM and Western medicine and the dialectical criteria of cold coagulation and blood stasis-type primary dysmenorrhea, 60 patients with primary dysmenorrhea were selected. According to the simple random method (lottery method), the 60 patients with cold coagulation and blood stasis-type primary dysmenorrhea were randomly assigned to the graphene AHP for warming uterus and relieving pain group (treatment group) and the placebo AHP group (control group, ingredients included buckwheat flour, honey, and excipient). One patient dropped out of each group during the implementation, that is, 29 patients each were actually included in the treatment group and the control group. The age and disease course of the patients in the experimental group and the control group were comparable, with no statistically significant difference, allowing for further research.

The experimental group and the control group were operated in the same manner as those for the qi stagnation and blood stasis-type primary dysmenorrhea.

After 3 courses of treatment, the effects were evaluated (including VAS, CMSS, and TCM symptom scores), and the results were statistically analyzed using SPSS26.0 software.

### (5) Comparison of VAS Between Groups

The comparison results of the VAS between groups were shown in Table 5.

TABLE 5
Comparison of VAS scores between two groups of patients/[M(Q)]

| | | First | Second | Third |
|---|---|---|---|---|
| | Before | menstrual | menstrual | menstrual |
| | treatment | cycle | cycle | cycle |
| Treatment group | 7 (2.00) | 4.83 ± 1.51 | 3.70 ± 1.70 | 2 (2.00) |
| Control | 7 (1.00) | 6.00 ± 1.67 | 6.07 ± 1.60 | 7 (3.50) |
| t (Z) | −0.919▴ | −2.800* | −5.452* | −5.495▴ |
| P | 0.358▴ | 0.007* | 0.000* | 0.000▴ |

Note:
the menstrual cycles before treatment and the third month of treatment did not conform to the normal distribution, so they were expressed by the median and four-digit range [M(Q)].
*indicates the t/P value obtained by two independent sample tests.
▴indicates the Z/P value obtained by the Mann-Whitney U test.

As shown in Table 5, for the first and two/three courses of treatment, the difference between the two groups was statistically significant. This indicates that treatment in the experimental group could significantly reduce the VAS scores of patients with cold coagulation and blood stasis-type primary dysmenorrhea.

### (6) Comparison of CMSS Between Groups

The comparison results between CMSS groups were shown in Table 6.

TABLE 6
Comparison of CMSS scores between
two groups of patients [M(Q)]

| | | First | Second | Third |
|---|---|---|---|---|
| | Before | menstrual | menstrual | menstrual |
| | treatment | cycle | cycle | cycle |
| Treatment group | 35 (22.00) | 23 (13.00) | 17 (13.00) | 12 (11.00) |
| Control | 38 (30.00) | 32 (30.00) | 34 (33.00) | 30 (33.5) |
| Z | −0.755 | −3.439 | −4.412 | −5.011 |
| P | 0.450 | 0.001 | 0.000 | 0.000 |

Note:
before treatment, the first menstrual cycle, the second menstrual cycle and the third menstrual cycle did not meet the normal distribution, so they were expressed by median and four-digit range [M(Q)].

As shown in Table 6, the differences between the experimental group and the control group were statistically significant in the first, second and third courses of treatment. This indicates that treatment in the experimental group could significantly reduce the CMSS scores of patients with cold coagulation and blood stasis-type primary dysmenorrhea.

### (7) Comparison of Dysmenorrhea TCM Symptom Scores Between Groups

The comparison results of dysmenorrhea TCM symptom scores between groups are shown in Table 7.

TABLE 7
Comparison of TCM symptom scores
of two groups of patients [M(Q)]

| | | First | Second | Third |
|---|---|---|---|---|
| | Before | menstrual | menstrual | menstrual |
| | treatment | cycle | cycle | cycle |
| Treatment group | 12 (4.00) | 10 (2.00) | 8 (2.00) | 4 (4.00) |
| Control | 12 (2.00) | 12 (3.00) | 12 (4.00) | 12 (8.00) |
| Z | −1.717 | −4.555 | −5.513 | −5.669 |
| P | 0.086 | 0.000 | 0.000 | 0.000 |

As shown in Table 7, the differences between the experimental group and the control group were statistically significant in the first, second and third courses of treatment. This indicated that treatment in the experimental group could significantly reduce the TCM symptom scores of patients with cold coagulation and blood stasis-type primary dysmenorrhea.

### (8) Overall Clinical Effective Rate

The comparison results of the overall clinical effective rate between the experimental group and the control group are shown in Table 8.

TABLE 8
Overall efficacy evaluation of two groups of patients

| | | | | | | Overall | | |
|---|---|---|---|---|---|---|---|---|
| | Number | | Markedly | | | effective | | |
| Group | of cases | Cure | effective | Effective | Ineffective | rate | Z | P |
| Treatment | 29 | 4 | 10 | 11 | 4 | 86.21% | −4.882 | 0.000 |
| group | | | | | | | | |
| Control | 29 | 0 | 0 | 9 | 20 | 31.03% | | |

Table 8 indicates that there was a high overall effective rate of graphene AHP for warming uterus and relieving pain in treating the cold coagulation and blood stasis-type primary dysmenorrhea.

### 3. Clinical Study of the Graphene AHP for Warming Uterus and Relieving Pain in Treating Adolescent Primary Dysmenorrhea

### (1) Diagnostic Criteria

### 1) Diagnostic Criteria in Western Medicine

The Western medicine diagnostic criteria for adolescent primary dysmenorrhea were the same as the diagnostic criteria for qi stagnation and blood stasis-type primary dysmenorrhea.

### 2) Diagnostic Criteria in TCM

The criteria were formulated with reference to the 2017 “TCM Gynecology” published by China TCM Publishing House: (i) periodic lower abdominal pain during menstruation or before and after menstruation, pain reaching the lumbosacral region, and even fainting; (ii) common in young unmarried women; (iii) excluding abdominal pain caused by pelvic organic diseases.

### (2) Efficacy Observation Indicators

### 1) VAS

The VAS adopted a 10-point scale line, where 0 represents no pain and 10 represents extreme pain.

### 2) CMSS

The CMSS was the same as that of qi stagnation and blood stasis-type primary dysmenorrhea.

### 3) Criteria for Judging Overall Efficacy

The criteria for judging overall efficacy was the same as that of qi stagnation and blood stasis-type primary dysmenorrhea.

### (3) Case Criteria

Inclusion criteria: (i) those who meet diagnostic standards of TCM and Western medicine for primary dysmenorrhea; (ii) females aged 10-19 years old who are not pregnant; (iii) menstrual regularity: the average menstrual cycle is 21-35 d, and the average menstrual period is 3-7 d; (iv) VAS score ≥4 points; (v) CMSS score >10 points; (vi) no history of drug allergy; (vii) voluntarily sign the informed consent form (those under 18 years of age must sign at the same time from their guardian); note: patients must meet the above 7 criteria at the same time to be included.

Exclusion criteria: (i) presence of concomitant organic uterine diseases; (ii) presence of concomitant serious primary diseases and malignant tumors such as heart, liver, kidney; (iii) those who are taking oral contraceptives or using the Mirena ring; (iv) those with severe mental illness, such as moderate to severe anxiety and depression; (v) those with a history of severe skin allergies and allergies to a variety of foods and drugs; (vi) those who are preparing to become pregnant or are in pregnancy or lactation period; (vii) those who do not comply with patching treatment. Note: patients who meet any of the above criteria need to be excluded.

Dropout and discontinuation criteria: (i) those who experience allergic reactions or other adverse events; (ii) those with poor compliance, disobedience, and failure to follow treatment time; (iii) those taking other therapeutic drugs or receiving other treatment methods (which will clearly affect the test results); (iv) those who are unblinded during the trial due to various reasons.

Exclusion criteria: (i) those who are mistakenly included due to misdiagnosis; (ii) those who do not receive treatment as scheduled after inclusion and have no evaluation records; (iii) those who receive other treatments or take other drugs (which will clearly affect the test results); (iv) participants in other clinical trials during the trial.

### (4) Case Grouping

According to the diagnostic criteria of dysmenorrhea of TCM and Western medicine and the dialectical criteria of dysmenorrhea, 60 patients aged 10-19 years with adolescent primary dysmenorrhea were selected. According to the simple random method (lottery method), the patients were numbered according to the order of enrollment, and were divided into graphene AHP for warming uterus and relieving pain group (treatment group) and the placebo AHP group (control group, ingredients included buckwheat flour, honey, and excipient) using random numbers generated by Excel. Two patients dropped out of the control group during the implementation, that is, 30 patients were actually included in the treatment group and 28 patients in the control group. The age and disease course of the patients in the experimental group and the control group were comparable, with no statistical significant difference, allowing for further research.

The experimental group and the control group were operated in the same manner as those in the qi stagnation and blood stasis-type primary dysmenorrhea.

After 3 courses of treatment, the effects were evaluated (including VAS, CMSS, and TCM symptom scores), and the results were statistically analyzed using SPSS26.0 software.

### (5) Comparison of VAS Between Groups

The comparison results of the VAS between groups were shown in Table 9.

TABLE 9
Comparison of VAS between groups

| | | After one | After two | After three |
|---|---|---|---|---|
| | Before | courses of | courses of | courses of |
| Group | treatment | treatment | treatment | treatment |
| Treatment | 5.97 ± 1.33 | 4.47 ± 1.48 | 3.53 ± 1.46 | 2.83 ± 1.62 |
| group | | | | |
| t value | | 7.225 | 11.439 | 13.706 |
| P value | | 0.000¹⁾ | 0.000¹⁾ | 0.000¹⁾ |
| Control | 5.89 ± 1.29 | 5.89 ± 1.26 | 5.64 ± 1.42 | 5.39 ± 1.75 |
| Z value | | 0.000 | −1.941 | −1.852 |
| P value | | 1.000²⁾ | 0.052²⁾ | 0.064²⁾ |
| t value | 0.215 | −3.942 | −5.580 | −5.784 |
| between | | | | |
| two groups | | | | |
| P value | 0.831 | 0.000³⁾ | 0.000³⁾ | 0.000³⁾ |
| between | | | | |
| two groups | | | | |

Note:
comparison within the groups, compared with this group before treatment:
¹⁾P < 0.05,
²⁾P > 0.05; comparison between groups:
³⁾P < 0.05.

As shown in Table 9, the differences between the experimental group and the control group were statistically significant in the first, second and third courses of treatment. This indicates that treatment in the experimental group could significantly reduce the VAS scores of patients with adolescent primary dysmenorrhea.

### (6) Comparison of CMSS Between Groups

The comparison results between CMSS groups were shown in Tables 10 and 11.

TABLE 10
Comparison of CMSS dysmenorrhea severity
scores between two groups in each period

| | | After one | After two | After three |
|---|---|---|---|---|
| | Before | courses of | courses of | courses of |
| Group | treatment | treatment | treatment | treatment |
| Treatment | 25.23 ± 7.06 | 19.67 ± 6.70 | 16.00 ± 6.30 | 12.33 ± 6.04 |
| group | | | | |
| t value | | 10.042 | 12.043 | 11.886 |
| P value | | 0.000¹⁾ | 0.000¹⁾ | 0.000¹⁾ |
| Control | 23.90 ± 5.09 | 23.32 ± 5.04 | 23.07 ± 5.33 | 21.79 ± 7.06 |
| t value | | 1.982 | 1.856 | 1.977 |
| P value | | 0.058²⁾ | 0.074²⁾ | 0.058²⁾ |
| t value | 0.824 | −2.334 | −4.596 | −5.486 |
| between | | | | |
| two groups | | | | |
| P value | 0.414 | 0.023³⁾ | 0.000³⁾ | 0.000³⁾ |
| between | | | | |
| two groups | | | | |

TABLE 11
Comparison of CMSS dysmenorrhea duration
scores between two groups in each period

| | | After one | After two | After three |
|---|---|---|---|---|
| | Before | courses of | courses of | courses of |
| Group | treatment | treatment | treatment | treatment |
| Treatment | 24.60 ± 7.83 | 19.50 ± 7.19 | 15.83 ± 6.82 | 11.97 ± 7.21 |
| group | | | | |
| t value | | 9.577 | 11.281 | 13.053 |
| P value | | 0.000 | 0.000 | 0.000 |
| Control | 24.10 ± 5.94 | 23.53 ± 5.94 | 23.39 ± 6.09 | 22.18 ± 7.42 |
| t value | | 1.706 | 1.795 | 2.016 |
| P value | | 0.100 | 0.084 | 0.054 |
| t value | 0.269 | −2.321 | −4.438 | −5.314 |
| between | | | | |
| two groups | | | | |
| P value | 0.789 | 0.024 | 0.000 | 0.000 |
| between | | | | |
| two groups | | | | |

Note:
comparison within the groups, compared with this group before treatment:
¹⁾P < 0.05,
²⁾P > 0.05; comparison between groups:
³⁾P < 0.05.

As shown in Tables 10 and 11, the differences between the experimental group and the control group were statistically significant in the first, second and third courses of treatment. This indicates that treatment in the experimental group could significantly reduce the CMSS scores of patients with adolescent primary dysmenorrhea.

### (7) Comparison of Overall Clinical Efficacy

TABLE 12
Comparison of overall effective rates between two groups after treatment

| | | Markedly | | | Overall | | |
|---|---|---|---|---|---|---|---|
| | Cure | effective | Effective | Ineffective | effective | | |
| Group | (case) | (case) | (case) | (case) | rate (%) | Z value | P value |
| Treatment | 1 | 7 | 17 | 5 | 83.33% | −4.453 | 0.000 |
| group | | | | | | | |
| Control | 0 | 0 | 8 | 20 | 28.57% | | |

Note:
Overall effective rate = (cure cases + markedly effective cases + effective cases)/n × 100%

Table 12 indicates that the graphene AHP for warming uterus and relieving pain had a high overall effective rate in treating the adolescent primary dysmenorrhea.

The above descriptions are merely preferred embodiments of the present disclosure. It should be noted that a person of ordinary skill in the art may further make several improvements and modifications without departing from the principle of the present disclosure, and such improvements and modifications should be deemed as falling within the protection scope of the present disclosure.

What is claimed is:

1. A graphene acupoint herbal patch (AHP) for warming uterus and relieving pain, comprising the following raw materials in parts by weight: 25 parts of a traditional Chinese medicine (TCM), 0.1 parts of graphene, 64 parts of hot melt adhesive, 2 parts of far-infrared ceramic powder, 3 parts of heat sensitizer, 3 parts of camphor, 3 parts of menthol; wherein the TCM comprises Rhizoma cyperi, Flos caryophylli, Rhizoma corydalis, Resina olibani, Myrrha, Radix angelicae sinensis, Radix angelicae dahuricae, Herba leonuri, Flos carthami, Fructus foeniculi, Ramulus cinnamomi, Rhizoma zingiberis, and Radix paeoniae alba in a mass ratio of 1:1:1:1:1:1:1:1:1:1:1:1:1; and
a method for preparing the graphene AHP for warming uterus and relieving pain comprises the following steps: crushing the TCM, and mixing the crushed TCM with the graphene to obtain a TCM-graphene mixture; and mixing a melted hot melt adhesive with the far-infrared ceramic powder, the heat sensitizer, the TCM-graphene mixture, the camphor, and the menthol, and applying a resulting mixture on a patch to obtain the graphene AHP for warming uterus and relieving pain.

2. A method for treating dysmenorrhea, comprising administering the graphene AHP for warming uterus and relieving pain according to claim 1 to a patient in need thereof.
